(19) United States
(12) Patent Application Publication
(10) Pub. No.: US 2025/0073143 A1
(43) Pub. Date: Mar. 6, 2025

(54) PERSONAL CARE COMPOSITIONS INCLUDING ANTIPERSPIRANT, DEODORANT, AND ASTRINGENT COMPOSITIONS

(71) Applicant: ELEMENTIS SPECIALTIES, INC., East Windsor, NJ (US)

(72) Inventors: Rey ORDIALES, East Windsor, NJ (US); Kimberly BURCH, East Windsor, NJ (US); Victoria CARDARELLI, East Windsor, NJ (US); Barbara M. SCHMIDT, East Windsor, NJ (US)

(21) Appl. No.: 18/955,716

(22) Filed: Nov. 21, 2024

Related U.S. Application Data
(63) Continuation-in-part of application No. PCT/US2024/031473, filed on May 29, 2024.
Application No. PCT/US2024/031473 is a continuation of application No. 18/203,953, filed on May 31, 2023.

(30) Foreign Application Priority Data
Aug. 23, 2023 (GB) 2312153.6

(51) Int. Cl. A61K 8/36; A61K 8/37; A61K 8/49; A61K 8/9789; A61Q 15/00

(52) U.S. Cl. CPC A61K 8/36; A61K 8/37; A61K 8/498; A61K 8/9789; A61Q 15/00; A61K 2800/48; A61K 2800/874

(57) ABSTRACT

A personal care composition comprising a personal care agent is described. The personal care agent comprises an optionally substituted benzenepropenoic acid. Also described is the optionally substituted benzenepropenoic acid that is optionally in combination with an ester of gallic acid. Methods and uses of the personal care composition for reducing or preventing perspiration, improving appearance, cleansing, controlling odor, or providing general aesthetics, are also described.

### CROSS-REFERENCE TO RELATED APPLICATIONS

This application is a continuation-in-part of International Application No. PCT/US2024/031473, filed May 29, 2024, which claims priority to U.S. patent application Ser. No. 18/203,953, filed May 31, 2023, and to United Kingdom Application No. GB 2312153.6, filed Aug. 8, 2023.

### FIELD OF THE INVENTION

The invention relates to personal care compositions, such as antiperspirant compositions, deodorant compositions, astringent compositions, and personal care compositions related thereto, including but not limited to, shampoo and bodywash compositions. The invention also relates to methods of reducing or preventing perspiration, masking or preventing body odor, skin toning or reducing the effects of skin aging, improving appearance, cleansing, and general aesthetics, as well as the use of certain natural or naturally obtainable ingredients, particularly phytochemicals, and/or aluminum-free synthetic/chemical functional equivalents, as personal care agents, such as antiperspirant, deodorant, and astringent agents.

### BACKGROUND

Personal care compositions, such as antiperspirant compositions, deodorant compositions, astringent compositions, and compositions related thereto, such as but not limited to shampoo and body wash compositions, are known in the art.

Antiperspirant compositions are a well-known type of product in the field of personal care and are used to eliminate or reduce perspiration or sweating, which is important for daily hygiene. Deodorant compositions are well-known in the field of personal care and are used to eliminate or mask unpleasant smells, such as body odor. Astringent compositions are well-known in the field of personal care and are used to tone or tighten skin to reduce the effects of skin aging. These compositions are available in a variety of forms for administration, such as solid sticks, roll-ons, wipes, aerosol sprays, pump sprays, gel formulations, lotion formulations, and the like.

The antiperspirant compositions generally contain an active ingredient, often referred to as an antiperspirant agent, which may narrow or block the ducts of sweat glands to reduce or prevent perspiration. The antiperspirant agent may also provide a deodorizing effect if it is able to mask odors or it may have antimicrobial activity.

Deodorant compositions generally contain an active ingredient, often referred to as a deodorant agent, which typically includes one or more of an antiseptic, anti-bacterial, anti-microbial, fragrance, perfume, antibiotic and/or fragrance component. Body odor is reduced by either masking, neutralizing or eliminating the odor. In addition, these deodorant agents may also function by eliminating the odor causing bacteria.

Astringent compositions generally contain an active ingredient, often referred to as an astringent agent, that restricts, lessens in size, shrinks, or constricts the number of bodily fluids that are released from the sweat ducts or bodily pores. Astringents provide a skin toning effect to reduce skin aging.

Antiperspirant agents, deodorant agents, astringent agents, as well as related personal care agents, such as but not limited to, shampoo and body wash agents, in common use generally include aluminum or aluminum-zirconium salts, such as aluminum chlorohydrate or aluminum zirconium tetrachlorohydrex gly (AZG). For example, with respect to antiperspirants, these salts precipitate in humid conditions on the skin and interact with proteins in the skin to form macromolecules, which plug pores, to reduce or prevent sweating.

However, there is a need in the art to design and implement personal care compositions and agents wherein the aluminum or aluminum-zirconium salts are substituted or combined with aluminum-free materials, which are natural materials, naturally derived or obtained materials, or synthetic/chemical functional equivalents thereof. For example, the search for alternative antiperspirant agents or compounds with similar functionalities has been ongoing to match or improve the sweat reduction properties of aluminum or aluminum zirconium salts in known antiperspirant agents. Similarly, the search for alternative agents or compounds with similar functionalities to deodorant and astringent agents as well as shampoo and body wash agents, has been ongoing to match or improve the deodorizing and toning properties, or cleansing properties, of aluminum or aluminum zirconium salts in known deodorants, and astringents, and related personal care compositions.

### SUMMARY OF THE INVENTION

In a first aspect, the invention provides a personal care composition, including but not limited to, an antiperspirant composition, a deodorant composition, or an astringent composition. The personal care composition includes a personal care agent (an active agent) including an optionally substituted benzenepropenoic acid represented by formula (I):

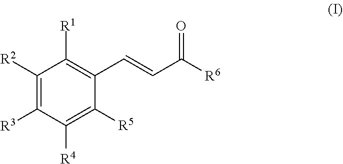

wherein each of R¹ to R⁵ is independently selected from hydrogen, hydroxy and C₁-C₆ alkoxy, and R⁶ is selected from hydrogen and hydroxy; and a carrier.

For example, an antiperspirant, a deodorant, or an astringent composition comprises an antiperspirant, deodorant, or astringent agent, respectively, wherein the antiperspirant, deodorant, or astringent agent comprises an aluminum-free active agent material or a blend thereof.

The first aspect of the invention also provides a personal care composition, including but not limited to an antiperspirant, a deodorant, or an astringent composition including a personal care agent, including but not limited to an antiperspirant, a deodorant, or an astringent agent, respectively, wherein the optionally substituted benzenepropenoic acid, includes comprises a natural material or source, e.g., phytochemical or natural extract, a material derived/derivable or obtained/obtainable therefrom, or an aluminum-free synthetic/chemical material that is an equivalent of the phytochemical or material derived or obtained from the phytochemical. In certain embodiments, the natural material or source is selected from a terpenoid, flavonoid, phenolic acid, phenol, quinone, and blends or combinations thereof. Non-limiting examples of the terpenoid include but are not limited to Genipin, geniposide, Aucubin, Cassane diterpenes, Soyasapogenol A, and blends or combinations thereof. Non-limiting examples of the flavonoid include but are not limited to Naringenin, Osajin and pomiferin, Emblicanins, Gallotannins, Gallocetechin, and blends or combinations thereof. Non-limiting examples of the phenolic acid include but are not limited to Gallic acid, Ellagic acid, and blends or combinations thereof. Non-limiting examples of the phenol include but are not limited to Arubtin, Oleuropein, and blends and combinations thereof. Non limiting examples of the quinones include but are not limited to Rhein, Emodin, Aloe-emodin, and blends and combinations thereof.

In a second aspect, the invention also provides a personal care composition, including but not limited to an antiperspirant, a deodorant, or an astringent composition comprising a personal care agent, including but not limited to an antiperspirant, a deodorant, or an astringent agent, respectively, wherein the agent (active agent) comprises an optionally substituted benzenepropenoic acid derived from a phytochemical.

In a third aspect, the invention provides a personal care composition, including but not limited to an antiperspirant, a deodorant, or an astringent composition comprising a personal care agent, including but not limited to an antiperspirant, a deodorant, or an astringent agent, respectively, wherein the agent (active agent) comprises (a) a natural material or source, (b) a material derived/derivable or obtained/obtainable therefrom, or (c) an aluminum-free synthetic/chemical material that demonstrates or imparts functional properties that are equivalent or substantially equivalent to (a) or (b).

The natural material comprises a phytochemical or natural extract and optionally comprises one or more of a terpenoid, flavonoid, phenolic acid, phenol, quinone, and blend or combination thereof. Optionally, the natural extract comprises cinnamon extract or a combination or blend of cinnamon extract and Emblica officinalis extract. In one embodiment, the natural extract is cinnamon extract, which optionally includes cinnamic acid, e.g., trans-cinnamic acid.

It has surprisingly been found that certain classes of phytochemicals or natural extracts, e.g., plant extracts, as well as materials derived/derivable or obtained/obtainable therefrom or aluminum-free synthetic/chemical functional equivalents thereof, possess personal care properties, such as but not limited to, antiperspirant, deodorant, and astringent properties; therefore, they are useful and effective as personal care agents, such as but not limited to, antiperspirant, deodorant, and astringent agents (active agents), particularly, in personal care products. These materials provide a comparable personal care effect, e.g., an antiperspirant, a deodorant, or an astringent effect, to the aluminum or aluminum-zirconium salts that are typically included in antiperspirant, deodorant, and astringent agents (active agents) in most commercially available personal care products. Thus, the phytochemicals or natural extracts, materials derived/derivable or obtained/obtainable therefrom, or aluminum-free synthetic/chemical functionally equivalents are included as personal care agents, e.g., antiperspirant, deodorant, or astringent agents (active agents), as substitutes for or in place of the aluminum or aluminum-zirconium salts. These materials, particularly the natural extracts and those derived/derivable therefrom, may also impart a pleasant odor to the personal care composition.

The fourth aspect of the invention relates to methods of providing a personal care effect, such as but not limited to, reducing or preventing perspiration, eliminating or masking body odor, toning or reducing the effects of skin aging, improving appearance, cleansing, and general aesthetics. The methods comprise applying a personal care composition in accordance with the first or second or third aspects of the invention, to skin.

The fourth aspect of the invention also includes the use of the related personal care compositions to reduce or prevent perspiration, eliminate or mask body odor, tone or reduce the effects of skin aging, improve appearance, cleanse, or general aesthetics.

A fifth aspect of the invention relates to the use of an optionally substituted benzenepropenoic acid for reducing or preventing perspiration, eliminating or masking body odor, toning or reducing the effects of skin aging, improving appearance, cleansing, or general aesthetics.

A sixth aspect of the invention relates to a method of preparing a personal care composition including preparing a personal care agent (active agent) including an optionally substituted benzenepropenoic acid represented by formula (I):

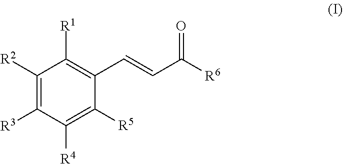

wherein each of R¹ to R⁵ is independently selected from hydrogen, hydroxy and C₁-C₆ alkoxy, and R⁶ is selected from hydrogen and hydroxy; and a carrier.

In certain embodiments, the optionally substituted benzenepropenoic acid includes a phytochemical, or a material derived or obtained from the phytochemical, or an aluminum-free synthetic/chemical material that is an equivalent of the phytochemical or material derived or obtained from the phytochemical. Optionally, the mixture further includes one or more materials selected from lubricant fatty alcohol, thickener, and modified starch.

In certain embodiments, the method includes mixing a natural material or source, e.g., phytochemical or natural extract, or a material derived/derivable or obtained/obtainable therefrom, or an aluminum-free synthetic/chemical functional equivalent, with an acceptable carrier. Optionally, the personal care composition additionally includes combining one or more of a lubricant, fatty alcohol, starch and thickener with the acceptable carrier to form a mixture; the mixture is heated to a temperature effective to melt solids present and a thickener is added, dispersing the thickener. A starch is then added, followed by addition of the a phytochemical or natural extract, or a material derived/derivable or obtained/obtainable therefrom, or an aluminum-free synthetic/chemical functional equivalent, as a personal care agent (active agent) for reducing or preventing perspiration, eliminating or masking body odor, toning or reducing the effects of skin aging, improving appearance, cleansing, or general aesthetics, and then cooling the mixture. The natural extract in this aspect is cinnamon extract or cinnamon extract and Emblica officinalis extract. It is preferred that the natural extract is cinnamon extract, which optionally or preferably includes trans-cinnamic acid. In certain embodiments, the materials derived from the natural extract, or the aluminum-free synthetic/chemical materials, demonstrate or impart equivalent or substantially equivalent functional properties as the cinnamon extract or cinnamon extract and Emblica officinalis extract. The phytochemical is selected from a terpenoid, flavonoid, phenolic acid, phenol, quinone, and combinations or blends thereof.

### DETAILED DESCRIPTION OF PREFERRED EMBODIMENTS

The following definitions apply to the disclosure herein, including all aspects of the invention and their embodiments.

The term “personal care” as used herein, whether used alone or in conjunction with another term, refers to a non-therapeutic use, such as a non-therapeutic, cosmetic use. This term refers to use for personal hygiene. The term “personal care” includes leave-on or rinse-off compositions and agents applied to the human body for improving appearance, cleansing, odor control, sweat control, toning, or general aesthetics. The personal care compositions and agents include the forms of a liquid, lotion, cream, foam, scrub, emulsion, and gel. Non-limiting examples include leave-on skin lotions and creams, shampoos, conditioners, shower gels, antiperspirants, deodorants, astringents, and the like. The term “skin” refers to skin on the face and body, such as, neck, chest, back, arms, underarms, hands, legs, buttocks, and scalp.

The compounds and intermediates described herein may be named according to the IUPAC (International Union for Pure and Applied Chemistry) or the CAS (Chemical Abstracts Service) nomenclature systems or by their commercial trade names. It should also be understood that any reference to substituted benzenepropenoic acid encompasses all stereoisomers (e.g., cis and trans isomers) of such compounds.

The term “phytochemical” as used herein refers to a chemical compound that is produced by a plant (e.g. through primary or secondary metabolism). In other words, a phytochemical is a naturally occurring, biologically active compound found in a plant. The phytochemical typically has biological activity that protects the plant from the environment, disease or a predator.

The term “flavonoid” as used herein refers to a compound having a 15-carbon core structure (e.g. skeleton) consisting of two phenyl rings (referred to as the “A” and “B” rings) and a heterocyclic ring (referred to as the “C” ring), which contains an oxygen atom. The term “flavonoid” is used in the conventional, restricted sense as referring only to those compounds with a C₆-C₃-C₆ carbon framework (e.g. core structure or skeleton) having the structure of a chromane or a 1-benzopyran (chromene), in which the fused benzene ring is designated as ring A and the 3,4-dihydro-2H-pyran or the pyran as ring C, along with a phenyl group (ring B) on ring C (see, for example, the “Nomenclature of flavonoids” by A. P. Rauter et al.—Reference 1). The general structure of a flavonoid with ring labelling is shown below.

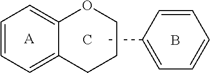

Depending on the position of the linkage of ring B to the chromane/1-benzopyran (chromene) moiety, the term “flavonoid” comprises, or consists of, the sub-classes of compounds known as 2-flavonoids, 3-flavonoids (e.g. also known as isoflavonoids) and 4-flavonoids (e.g. also known as neoflavonoids).

The term “2-flavonoid” as used herein refers to a compound having a core structure or skeleton as represented by formula (A-1).

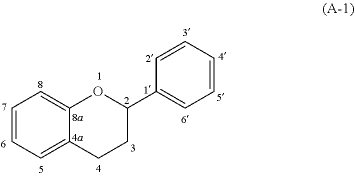 [SMILES: c1ccc(C2CCc3ccccc3O2)cc1]

In formula (A-1), the atoms of the core structure are numbered in accordance with the IUPAC numbering scheme for this sub-class of compounds.

For the avoidance of doubt, formula (A-1) represents the core structure or skeleton of a 2-flavonoid. Rings A to C of the core structure may include substituents. Ring C may include a carbon-carbon double bond (in addition to the unsaturation arising from the fused phenyl group, which is ring A) and/or a carbonyl group.

In general, the term “2-flavonoid” includes a flavan, a flavone or a flavanol.

When ring C in formula (A-1) does not include a carbon-carbon double bond or a carbonyl group, then formula (A-1) represents a flavan. The term “flavan” refers to a compound with a 2-phenyl-3,4-dihydro-2H-1-benzopyran skeleton.

When ring C in formula (A-1) is substituted with a hydroxy group, such as at the 3-position and/or the 4-position of ring C, then the 2-flavonoid is a flavanol.

The core structure or skeleton of the flavanol may be a flavan-3-ol (as represented by formula (A-2)), a flavan-4-ol (as represented by formula (A-3)) or a flavan-3,4-diol (as represented by formula (A-4)).

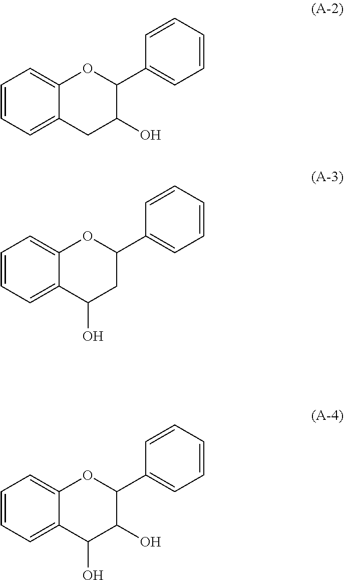 [SMILES: OC1Cc2ccccc2OC1c1ccccc1 ; OC1CC(c2ccccc2)Oc2ccccc21 ; OC1c2ccccc2OC(c2ccccc2)C1O]

When ring C in formula (A-1) includes a carbonyl group, such that the compound is derived from a 2-phenyl-2,3-dihydro-4H-1-benzopyran-4-one skeleton, then the 2-flavonoid is a flavanone, specifically a flavan-4-one (e.g. as represented by formula (A-5)).

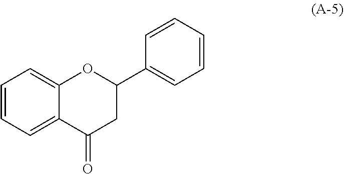 [SMILES: O=C1CC(c2ccccc2)Oc2ccccc21]

It is preferred that the core structure or skeleton of the 2-flavonoid is not an anthocyanidin (also known as an anthocyanin). An anthocyanidin is a compound derived from a flavylium (2-phenyl-1) 4-benzopyran-1-ylium or 2-phenylchromenylium) ion.

The term “glucogallin” as used herein refers to the following compound.

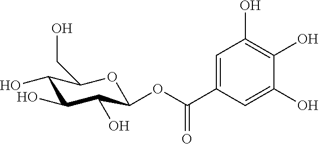

Glucogallin is typically present in a natural extract of Emblica officinalis. Thus, glucogallin may be obtained or is obtainable from Emblica officinalis extract.

The term “saberry” or “saberry extract” as used herein is synonymous with Emblica officinalis or Emblica officinalis extract, respectively, and refers to fruits or an extract of fruits of Emblica officinalis (synonymous with Phyllanthus emblica), which is more commonly known as amla berry or Indian gooseberry.

As used herein by itself or in conjunction with another term or terms, “substituted” indicates that a hydrogen atom on a molecule has been replaced with a different atom or group of atoms. The atom or group of atoms replacing the hydrogen atom is a “substituent.” It should be understood that the terms “substituent”, “substituents”, “moiety”, “moieties”, “group”, or “groups” refer to substituent(s).

The various hydrocarbon-containing moieties provided herein may be described using a prefix designating the minimum and maximum number of carbon atoms in the moiety, namely “Cₐ-C_{b}”. For example, Cₐ-C_{b} alkyl indicates an alkyl moiety having the integer “a” to the integer “b” number of carbon atoms, inclusive.

As used herein, the term “alkyl”, whether used alone or in conjunction with another term, refers to a branched or unbranched saturated hydrocarbon chain, unless specified otherwise. An alkyl group is typically unsubstituted, unless indicated otherwise. An alkyl group may have 1 to 6 carbon atoms, such as 1 to 4 carbon atoms. Representative examples include, but are not limited to, methyl, ethyl, n-propyl, i-propyl, n-butyl, i-butyl, s-butyl, t-butyl, n-pentyl, n-hexyl.

As used herein, the term “alkoxy” refers to an alkyl-oxy group, wherein the alkyl group is as previously defined. An alkoxy group is typically unsubstituted, unless specified otherwise. Representative examples include —OMe, —OEt, —OnPr, —OiPr.

The term “deodorant” as used herein refers to an agent, material or composition that masks, neutralizes, eliminates or inhibits the formation of unpleasant body odors and/or provides an anti-bacterial effect, e.g., eliminates odor causing bacteria.

The term “natural extract” as used herein refers to a product that is derived (or derivable) or obtained (or obtainable) from natural sources, such as plants, fruits, vegetables, botanicals, herbs, and spices. The process of making an extract involves combining raw materials with a mixture of water and ethanol while heating them. After heating, the solids and liquids are separated, and the solids are concentrated into an extract.

Amounts of a material, such as a compound, composition or an ingredient, are typically defined in terms of the wt % of the composition.

The term “about” when used herein in conjunction with a measurable value encompasses reasonable variations of the value, for instance, to allow for experimental error in the measurement of said value.

As used in the present disclosure, the term “comprises” has an open meaning, which allows other, unspecified features to be present. This term embraces, but is not limited to, the semi-closed term “consisting essentially of” and the closed term “consisting of”. Unless the context indicates otherwise, the term “comprises” may be replaced with either “consisting essentially of” or “consists of”. The term “consisting essentially of” may also be replaced with “consists of”.

For the avoidance of doubt, the expression “consists essentially of” as used herein limits the scope of a feature to include the specified materials, and any other materials or steps that do not materially affect the basic and novel characteristics of that feature, such as, for example, minor impurities. The expression “consists essentially of” embraces the expression “consisting of”.

### Personal Care Compositions and Agents

The invention relates to the use of natural materials, sources or products or those that are naturally derived/derivable or obtained/obtainable therefrom or aluminum-free synthetic/chemical materials that demonstrate or impart equivalent or substantially equivalent functional properties as the natural or naturally-derived/obtained products, as personal care compositions and agents (active agent or ingredient).

The personal care compositions and agents are for providing a personal care effect. The personal care compositions and agents are for reducing or preventing perspiration, eliminating or masking body odor, toning or reducing the effects of skin aging, improving appearance, cleansing, or general aesthetics. In certain embodiments, an antiperspirant agent is an active ingredient for reducing or preventing perspiration in the antiperspirant composition of the invention, a deodorant agent is an active ingredient for eliminating or masking body odor, and an astringent agent is an active ingredient for toning or reducing the effect of skin aging.

Perspiration is a physiological body function that helps to control and preserve body temperature at approximately 37° C. Sweat is produced by sweat glands, which are cutaneous annexes composed by an outer layer of myoepithelial contractible cells and an inner layer of secreting cells. When the body temperature increases, the sweat glands are activated by a signal sent by the brain to produce sweat. Besides preserving stability of body temperature, sweat also humidifies skin and releases toxins. Excess transpiration can cause discomfort, such as underarm bad odor and lesions.

In certain embodiments, antiperspirants work by limiting the quantity of sweat released by the skin through the ducts of sweat glands by forming a buffer.

The personal care agent of the invention is for external use (e.g. on the human body).

The personal care composition or the personal care agent is for application to a surface of a body, particularly but not limited to the skin.

The personal care agent includes an optionally substituted benzenepropenoic acid. The personal care agent includes or consists essentially of an optionally substituted benzenepropenoic acid. In certain embodiments, the optionally substituted benzenepropenoic acid is a trans-benzenepropenoic acid.

The optionally substituted benzenepropenoic acid is represented by formula (I):

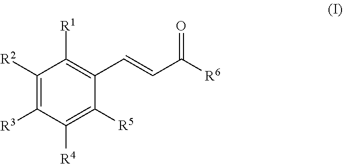

wherein each of R¹ to R⁵ is independently selected from hydrogen, hydroxy and C₁-C₆ alkoxy, and R⁶ is selected from hydrogen and hydroxy. The hydroxy (—OH) and the C₁-C₆ alkoxy groups are the optional substituents aforementioned (i.e., “optionally substituted”).

In certain embodiments, the optionally substituted benzenepropenoic acid includes a phytochemical, or a material derived or obtained from the phytochemical, or an aluminum-free synthetic/chemical material that is an equivalent of the phytochemical or material derived or obtained from the phytochemical. In certain embodiments, benzenepropenoic acid shows excellent personal care activity, such as but not limited to, antiperspirant activity.

The C₁-C₆ alkoxy is either ethoxy or methoxy. Optionally, the C₁-C₆ alkoxy is methoxy. In certain embodiments, at least three of R¹ to R⁵ are hydrogen. Optionally, two adjacent groups of R¹ to R⁵ is each independently selected from hydroxy and C₁-C₆ alkoxy. Optionally, (i) R¹ and R² or (ii) R² and R³ is each independently selected from hydroxy and C₁-C₆ alkoxy. Optionally, R¹, R⁴ and R⁵ are hydrogen, and each of R² and R³ is independently selected from hydroxy and C₁-C₆ alkoxy.

In certain embodiments, the optionally substituted benzenepropenoic acid is selected from cinnamic acid and its isomers, o-coumaric acid, m-coumaric acid, p-coumaric acid, caffeic acid, ferulic acid, 5-hydroxyferulic acid and sinapic acid. Optionally, the optionally substituted benzenepropenoic acid is caffeic acid. Optionally, the optionally substituted benzenepropenoic acid is trans-cinnamic acid. Optionally, the trans-cinnamic acid is present in cinnamon extract.

In certain embodiments, the personal care agent further includes an ester of gallic acid. The ester of gallic acid is selected from a flavonoid ester of gallic acid and a sugar ester of gallic acid. Optionally, the ester of gallic acid is a sugar ester of gallic acid, such as but not limited to glucogallin, and/or β-glucogallin. Optionally, the ester of gallic acid is a flavonoid ester of gallic acid, such as but not limited to a flavan-3-ol ester of gallic acid.

The flavan-3-ol ester of gallic acid includes the following structural moiety:

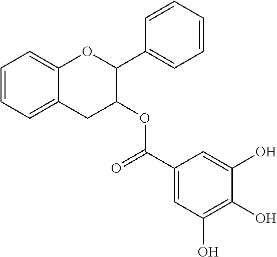 [SMILES: O=C(OC1Cc2ccccc2OC1c1ccccc1)c1cc(O)c(O)c(O)c1]

The term “moiety” is used because the above structure may contain one or more substituents on the 2-flavonoid skeleton or core structure.

Optionally, the flavan-3-ol ester of gallic acid is selected from catechin gallate, epicatechin gallate, epigallocatechin gallate and a mixture of two or more thereof. Optionally, the flavan-3-ol ester of gallic acid is epigallocatechin gallate. Optionally, the flavan-3-ol ester of gallic acid is selected from catechin gallate, epicatechin gallate and a mixture thereof. In certain embodiments, cinnamon extract contains a mixture of catechin gallate and epicatechin gallate.

The personal care agent includes an optionally substituted benzenepropenoic acid, optionally trans-cinnamic acid, and an ester of gallic acid. Further, the personal care agent is a combination or a mixture of the optionally substituted benzenepropenoic acid and the ester of gallic acid. Furthermore, the personal care agent includes or consists essentially of a natural material or source, such as, phytochemical or natural extract, a material derived/derivable or obtained/obtainable therefrom, or an aluminum-free synthetic/chemical material that demonstrates or imparts equivalent or substantially equivalent functional properties as the natural material and/or the material derived or obtained therefrom.

In certain embodiments, the natural extract is cinnamon extract or cinnamon extract and Emblica officinalis extract, such as a mixture of cinnamon extract and Emblica officinalis extract. The natural extract is preferably cinnamon extract, wherein, optionally, trans-cinnamic acid is derived/derivable or obtained/obtainable therefrom. Optionally, the natural material and/or the material derived or obtained therefrom is an aluminum-free synthetic/chemical material that demonstrates or imparts equivalent or substantially equivalent functional properties as the cinnamon extract or cinnamon extract and Emblica officinalis extract, or trans-cinnamic acid.

In certain embodiments, the personal care agent includes trans-cinnamic acid, Emblica officinalis extract and mixtures or blends thereof, and is absent of aluminum or aluminum-zirconium salts. In certain embodiments, the personal care agent does not include aluminum chlorohydrate or aluminum zirconium tetrachlorohydrex gly. Optionally, the personal care agent does not include aluminum. Optionally, the personal care agent does not include zirconium.

In certain embodiments, the personal care agent is one or more of an anti-oxidant, a protein coagulant, a preservative, and an anti-bacterial agent.

In certain embodiments, the personal care agent includes from 1.5 wt. % to 10.0 wt. % based on the total weight of the personal care composition. Optionally, the personal care agent is 1.5 wt. % of cinnamon extract and 1.5 wt. % Emblica officinalis extract. Optionally, the personal care agent is 3.0 wt. % of cinnamon extract. Optionally, the personal care agent is 3.0 wt. % of trans-cinnamic acid. Optionally, the personal care agent is 3.0 wt. % Emblica officinalis extract. Optionally, the personal care agent is 1.5 wt. % of trans-cinnamic acid and 1.5 wt. % of Emblica officinalis extract.

In certain embodiments, the personal care composition further includes cyclomethicone, modified starch, phenyl trimethicone, butyl ether, fumed silica, hydrogenated castor oil and stearyl alcohol.

The personal care agent is effective to reduce or prevent perspiration, and/or eliminate or mask body odor, and/or tone or reduce effects of skin aging.

In certain embodiments, the natural extract in the personal care agent present in the personal care composition includes cinnamon extract and optionally Emblica officinalis extract, such as a mixture of cinnamon extract and Emblica officinalis extract. Optionally, the natural extract is cinnamon extract, optionally cinnamon extract only, and optionally trans-cinnamic acid is derived/derivable or obtained/obtainable therefrom. In certain embodiments, the cinnamon extract includes one or more 2-flavonoids. Optionally, at least one of the one or more 2-flavonoids is a flavanol, optionally a flavan-3-ol. Optionally, the one or more 2-flavonoids is selected from afzelechin, epiafzelechin, catechin, epicatechin, gallocatechin, epigallocatechin or a combination of two or more thereof. Optionally, the one or more 2-flavonoids is a flavanone, preferably a flavan-4-one. Optionally, the flavan-4-one is rutin.

In certain embodiments, the cinnamon extract includes cinnamic acid, optionally trans-cinnamic acid.

In certain embodiments, the natural extract includes Emblica officinalis extract. Optionally, the Emblica officinalis extract includes glucogallin, optionally β-glucogallin.

In certain embodiments, the natural extract is one or more of an anti-oxidant, a protein coagulant, a preservative, and an anti-bacterial agent.

Accordingly, optionally, the natural extract is substitutable by a material that is naturally derived/derivable or obtained/obtainable therefrom or an aluminum-free synthetic/chemical material that demonstrates or imparts equivalent or substantially equivalent functional properties.

Without wishing to be bound by theory, the personal care agents of the invention, whether present as individual compounds or in the form of a natural material or source, e.g., phytochemical, or material derived/derivable or obtained/obtainable therefrom, e.g., natural extract, or an aluminum-free synthetic/chemical material that demonstrates or imparts equivalent or substantially equivalent functional properties, may provide a personal care effect, such as but not limited to, plugging the pores of sweat glands by causing protein aggregation and/or by binding to specific receptors in the wall of the sweat duct.

It has surprisingly been found that cinnamic acid, e.g., trans-cinnamic acid, has a personal care effect, such as but not limited to, an antiperspirant effect. It is believed that this compound causes coagulation or agglomeration of proteins.

Typically, the personal care agent including the natural material or source, material derived/derivable or obtained/obtainable therefrom, e.g., the natural extract, or an aluminum-free synthetic/chemical material functional equivalent thereof, has at least a 20% sweat reduction in at least 50% subjects tested, as measured according to US FDA Guidelines (Reference 3). The term “subjects tested” is synonymous with the “target population” mentioned in the US FDA Guidelines.

In general, the personal care agent including the natural material or source, material derived/derivable or obtained/obtainable therefrom, or aluminum-free synthetic/chemical material, is a deodorizing agent, optionally a deodorant.

Additionally, or alternatively, the personal care agent including the natural material or source, material derived/derivable or obtained/obtainable therefrom, or aluminum-free synthetic/chemical material has an astringent effect, a toning effect and/or an anti-aging effect on skin. In addition, or in the alternative, the personal care agent including the natural material or source, material derived/derivable or obtained/obtainable therefrom, or aluminum-free synthetic/chemical material has an improving appearance effect, cleansing effect, or general aesthetics effect.

### Personal Care Compositions

In certain embodiments, the personal care agent as defined above is the sole personal care agent in the personal care composition.

In general, the personal care composition does not comprise aluminum chlorohydrate or aluminum zirconium tetrachlorohydrex gly. It is preferred that the personal care composition does not comprise an aluminum-containing personal care agent (active agent or ingredient), more preferably the personal care agent does not include aluminum, or optionally does not include zirconium, or optionally does not include aluminum and zirconium.

Additionally, or alternatively, in certain embodiments, the personal care composition does not comprise 4-hydroxybenzaldehyde. In certain embodiments, the personal care composition does not comprise (i) a benzaldehyde or a benzaldehyde-derivative compound of formula I:

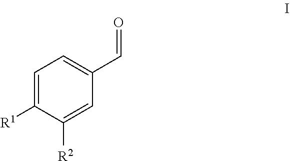

wherein R¹ is a residue selected from the group consisting of H, CH₂, CH₃, OH, and OCH₃, wherein R² is a residue selected from the group consisting of H, OH, OCH₃, and OCH₂O, and wherein if R² is OCH₂O, then R¹ is CH₂ and/or forms a bond with R²; and/or (ii) benzaldehyde, 4-methylbenzaldehyde, heliotropine, vanillin, 4-hydroxybenzaldehyde, 3-hydroxybenzaldehyde, 4-methoxybenzaldehyde, 3-methoxybenzaldehyde, or 3-hydroxy-4-methoxybenzaldehyde (isovanillin).

The personal care composition includes an effective amount of the personal care agent including the natural material or source, e.g., phytochemical or natural extract, or the material derived/derivable or obtained/obtainable therefrom, or an aluminum-free synthetic/chemical functional equivalent thereof. The effective amount is an amount that provides a personal care effect. Optionally, the effective amount is an amount that reduces or prevents perspiration, masks or prevents body odor and/or odor-causing bacteria, tone skin or reduce the effects of skin aging, improve appearance, cleanse, or provide general aesthetics.

The personal care composition includes a total amount of from about 0.1 to about 25.0 wt. % of the personal care agent, or from about 0.5 to about 15.0 wt. %, or from about 1.0 to about 10.0 wt. %, such as from about 1.0 to about 5.0 wt. % or from about 1.5 to about 10.0 wt. % of the personal care agent, based on total weight of the personal care composition.

Optionally, the personal care composition includes a total amount of about 0.1 to about 4.0 wt. % of the personal care agent, such as about 0.5 to about 4.0 wt. %, or from about 1.0 to 4.0 wt. %, such as about 1.5 to about 3.5 wt. % or about 1.5 to about 3.0 wt. % of the personal care agent, based on total weight of the personal care composition.

In certain embodiments, the personal care agent includes, or consists essentially of, an optionally substituted benzenepropenoic acid, such as trans-cinnamic acid, and the personal care composition includes the optionally substituted benzenepropenoic acid (e.g. trans-cinnamic acid) in a total amount from about 1.0 to about 20.0 wt % (e.g., about 3.0 to about 20.0 wt. %), or from about 1.5 to about 15.0 wt. % (e.g., about 5.0 to 15.0 wt. %), or 1.5 to about 10.0 wt. %, or from about 1.5 to about 3.0 wt. %, or from about 2.0 to about 10.0 wt. %, based on total weight of the personal care composition.

The personal care composition typically comprises a total amount of from about 5.0 to about 40.0 wt % of the natural material or source, e.g., phytochemical or natural extract, or the material derived or obtainable therefrom, or an aluminum-free synthetic/chemical material, or from about 10.0 to about 30.0 wt. %, from about 15.0 to about 25.0 wt. %, or from about 1.5 to about 15.0 wt. % (e.g., about 5.0 to 15.0 wt. %), or 1.5 to about 10.0 wt. %, or from about 1.5 to about 3.0 wt. %, or from about 2.0 to about 10.0 wt. %, of the natural material or source, e.g., phytochemical, or the material derived or obtainable therefrom, e.g., the natural extract, or an aluminum-free synthetic/chemical material, based on total weight of the personal care composition.

The amount of natural extract that is included in the personal care composition depends on the composition of the natural material or source, particularly the type and amount of ingredients that are present in the natural material or source, or the material derived or obtainable therefrom, or an aluminum-free synthetic/chemical material.

In general, the personal care composition is a cosmetic personal care composition, particularly for personal care. The personal care composition is typically for topical use. In certain embodiments, the personal care composition is a topical personal care composition.

The personal care composition is for topical application to skin, preferably a surface of the skin. In certain embodiments, the personal care composition is formulated for a specific mode of topical application.

Typically, the personal care composition comprises a topically acceptable carrier. The topically acceptable carrier will depend on the type of formulation and its mode of topical application.

In certain embodiments, the topically acceptable carrier is water, an alcohol, an ether, an oil, a wax or a combination thereof.

In certain embodiments, the topically acceptable carrier is water or combination (e.g., mixture or blend) of water and oil, such as when the personal care composition is in the form of a gel, a paste, a liquid, a lotion or a foam.

In certain embodiments, the topically acceptable carrier is, or includes, an alcohol. In certain embodiments, the alcohol is a mono-alcohol or a polyhydric alcohol (e.g., an alcoholic compound having two or more hydroxy groups).

In certain embodiments, when the alcohol is a mono-alcohol, the mono-alcohol is selected from ethanol or a fatty alcohol. In certain embodiments, the fatty alcohol has a straight chain length of 6 to 18 carbon atoms. It is preferred that the fatty alcohol is stearyl alcohol.

In certain embodiments, when the alcohol is a polyhydric alcohol, the polyhydric alcohol is a polymeric alcohol or a monomeric alcohol. In certain embodiments, the polymeric alcohol is a polyalkylene glycol, such as PEG or PPG. In certain embodiments, the monomeric alcohol is ethylene glycol, propylene glycol, glycerol or sugar, such as glucose, sorbitol or xylitol.

In certain embodiments, the topically acceptable carrier is, or includes, an ether; optionally, PPG-14 butyl ether.

In certain embodiments, the topically acceptable carrier is, or includes, an oil; optionally, hydrogenated castor oil.

In certain embodiments, the personal care composition is provided as, or included in, a deodorant, optionally, a body deodorant for personal hygiene purposes.

In certain embodiments, the personal care composition is in a form selected from a cream, a gel, a paste, a liquid, a lotion, a foam, an emulsion, a scrub or a powder.

Typically, the personal care composition is a leave-on composition. In certain embodiments, the leave on composition is applied to skin, or a surface of the skin, and left on for a period of time (e.g., from 1 minute to 24 hours), after which it may be wiped or rinsed off with water, usually during the regular course of washing.

In certain embodiments, when the personal care composition is in the form of a liquid, the liquid is a water-in-oil emulsion or an oil-in-water emulsion.

In certain embodiments, when the personal care composition is in the form of a liquid, the liquid is a sprayable liquid.

In certain embodiments, the personal care composition is for delivery as an aerosol. Optionally, the topically acceptable carrier comprises a propellant.

In certain embodiments, the personal care composition comprises a lubricant. Optionally, the lubricant is selected from a cyclomethicone, a dimethicone, a phenyltrimethicone or an ethyl methicone. Optionally, the lubricant is a cyclomethicone and/or phenyltrimethicone.

In certain embodiments, the personal care composition of the invention additionally comprises a natural oil. Optionally, the natural oil is included for skin care benefits.

Typically, the natural oil comprises an unsaturated fat, such as a polyunsaturated fat (e.g. a triglyceride derived from linoleic acid) and/or a monounsaturated fat (e.g. a triglyceride derived from oleic acid).

In certain embodiments, the personal care composition of the invention comprises a diluent such as water, liquid or solid emollients, solvents, humectants, thickeners and powders. Examples of each of these types of vehicle, which can be used singly or as mixtures of one or more vehicles are as follows:

Emollients, such as stearyl alcohol, glyceryl monoricinoleate, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils such as dimethylpolysiloxane, di-n-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isostearic acid, palmitic acid, isopropyl linoleate, lauryl lactate, myristyl lactate, decyl oleate and myristyl myristate;

Solvents, such as ethyl alcohol, isopropanol, acetone, ethylene glycol monoethyl ether, diethylene glycol monobutyl ether and diethylene glycol monoethyl ether; and

Powders, such as chalk, talc, fullers earth, kaolin, starch, gums, colloidal silica sodium polyacrylate, tetra alkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose and ethylene glycol monostearate.

In certain embodiments, the cosmetically acceptable base is from 10 to 99.9%, preferably from 50 to 99% w/w of the composition and, in the absence of other cosmetic adjuncts, forms the balance of the composition.

In certain embodiments, the personal care composition further comprises an antioxidant ingredient. Optionally, the antioxidant ingredient is included to chemically stabilize the personal care agent or any other natural ingredient that may be present, such a natural oil, particularly a natural oil comprising a polyunsaturated fat and/or a monounsaturated fat.

In certain embodiments, the antioxidant ingredient is selected from ascorbic acid, butyl hydroxy toluene, pentaerythrityl tetra-di-t-butyl hydroxyhydrocinnamate and tris-(tetramethylhydroxypiperidinol) citrate.

In certain embodiments, the personal care composition includes a fragrance.

In certain embodiments, the personal care composition includes a thickener. Optionally, the thickener is fumed silica or a cellulose ether, such as hydroxypropyl cellulose, hydroxypropyl methyl cellulose, hydroxyethyl cellulose, carboxymethyl cellulose, cetyl hydroxyethyl cellulose, hydroxybutyl methyl cellulose, methyl hydroxyethyl cellulose, clay, or xanthan gum. It is preferred that the thickener is fumed silica. Suitable clays include, but are not limited to, hectorite, bentonite, laponite, magnesium aluminum silicate, montmorillonite, kaolin, and blends and combinations thereof.

In certain embodiments, the personal care composition includes a surfactant. The surfactant may be an anionic surfactant, a cationic surfactant, a non-ionic surfactant or an amphoteric surfactant.

In certain embodiments, the personal care composition includes a personal care ingredient. Optionally, the personal care ingredient is a skin care agent, such as an emollient, a humectant, a skin barrier promoter or a skin appearance modifier.

Optionally, the personal care composition comprises a wide range of other optional components, such as a binder, a biological additive, a buffering agent, a rheology modifier or modified starch, a colorant, a fragrance, a conditioner, an exfoliating agent, a pH adjuster, a preservative, a skin soothing agent or a skin healing agent.

It is preferred that the personal care composition comprises a preservative and optionally one or more components selected from a binder, a biological additive, a buffering agent, a rheology modifier or modified starch, a colorant, a fragrance, a conditioner, an exfoliating agent, a pH adjuster, a skin soothing agent and a skin healing agent.

Typically, the personal care composition has at least a 20% sweat reduction in at least 50% subjects tested, as measured according to US FDA Guidelines.

In certain embodiments, the personal care composition is a deodorant composition, or an astringent composition, or a shampoo composition, or a body wash composition.

In certain embodiments, the personal care composition is manufactured by conventional methods known in the art.

### Methods and Uses

The method or uses of the invention are non-therapeutic.

A method of preparing the personal care composition includes preparing a mixture, including a personal care agent, including an optionally substituted benzenepropenoic acid represented by formula (I):

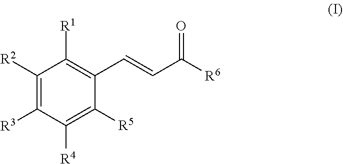

wherein each of R¹ to R⁵ is independently selected from hydrogen, hydroxy and C₁-C₆ alkoxy, and R⁶ is selected from hydrogen and hydroxy; and a carrier.

In certain embodiments, the optionally substituted benzenepropenoic acid includes a phytochemical, or a material derived or obtained from the phytochemical, or an aluminum-free synthetic/chemical material that is an equivalent of the phytochemical or material derived or obtained from the phytochemical. Optionally, the mixture further includes one or more materials selected from lubricant, fatty alcohol, thickener, rheology modifier and/or modified starch, e.g., Dry Flow PC. Optionally, the step of preparing the mixture, includes combining the lubricant, fatty alcohol and acceptable carrier to form a first mixture; heating the first mixture to form a heated first mixture; adding thickener to the heated first mixture to form a second mixture; and adding rheology modifier and/or modified starch to the second mixture, followed by adding the personal care agent.

The personal care composition of the invention is for use as a cosmetic, particularly for personal care.

The personal care composition or the personal care agent is applied to skin, preferably topically applied to skin. The personal care composition or the personal care agent is applied to a surface of the skin. In general, the skin is of a mammal, preferably a human.

Typically, the personal care composition or the personal care agent is applied to an area of skin where perspiration, odor or skin aging, occurs or an improved aesthetic effect or appearance is desired. For the antiperspirant, preferably, an area of the skin where there are sweat glands, such as apocrine sweat glands or eccrine sweat glands, preferably apocrine sweat glands. Optionally, the area of skin is on a thigh (e.g. inner thigh), the groin, the back, the upper lip, an armpit, the forehead, the knee (e.g. the back of the knee), the neck (e.g. back of the neck), the buttock, the chest, the scalp, feet or the hand.

In certain embodiments, the personal care composition or the personal care agent is applied by a direct body contact applicator, such as a roll-on lotion or a solid stick product. Alternatively, the personal care composition or the personal care agent is applied by a non-contact applicator, such as by spraying onto the skin. Optionally, the non-contact applicator is an aerosol spray, a pump spray or a squeeze spray.

In certain embodiments, the personal care agent, the natural material or source, or the material derived or obtainable therefrom, e.g., the natural extract, or an aluminum-free synthetic/chemical material, and/or the personal care composition is for use as a deodorant.

### EXAMPLES

The invention will now be illustrated by the following non-limiting examples.

### Examples 1 to 3

Formulation examples 1 to 3 shown in Table 1 were prepared.

TABLE 1

| | Formulation (F) No. | | |
|---|---|---|---|
| Ingredient | 1 | 2 | 3 |
| Natural extract | E1 | E1 + E2 | E3 |
| Cyclomethicone | ✓ | ✓ | ✓ |
| Aluminum starch | ✓ | ✓ | ✓ |
| succinate | | | |
| Phenyltrimethicone | ✓ | ✓ | ✓ |
| PPG-14 butyl ether | ✓ | ✓ | ✓ |
| Fumed silica | ✓ | ✓ | ✓ |
| Hydrogenated | ✓ | ✓ | ✓ |
| castor oil | | | |
| Stearyl alcohol | ✓ | ✓ | ✓ |
| 3-phenoxyethanol- | ✓ | ✓ | ✓ |
| 1,2-propanediol | | | |

In Table 1 above, E1 is cinnamon extract; E2 is Emblica officinalis extract; and E3 is trans-cinnamic acid. The extracts were obtained from Sabinsa Corporation. Each natural extract was included in an amount in each formulation to provide the same amount of antiperspirant agent across the formulations.

The amount of cinnamon extract in Formulation 1 was 3 wt %. The amount of cinnamon extract in Formulation 2 was 1.5 wt % and the amount of Emblica officinalis (saberry) extract in Formulation 2 was 1.5 wt %. The amount of trans-cinnamic acid in Formulation 3 was 3 wt %.

The amount for each of cyclomethicone, aluminum starch succinate, phenyltrimethicone, PPG-14 butyl ether, fumed silica, hydrogenated castor oil, stearyl alcohol, and 3-phenoxyethanol-1,2-propanediol was the same in all of the formulations (1, 2 and 3), as follows: 63.32 wt. % cyclomethicone, 8.93 wt. % aluminum starch octenylsuccinate, 7.66 wt. % phenyl trimethicone, 6.38 wt. % PPG-14 butyl ether, 3.83 wt. % fumed silica, 3.83 wt. % hydrogenated castor oil, 2.55 wt. % stearyl alcohol, and 0.5 wt. % 3-phenoxyethanol-1,2-propanediol.

### Comparative Examples

The formulations shown in Table 2 were prepared to provide a comparison to the natural extract formulations. No natural extracts were included in the below formulations.

TABLE 2

Comparative Formulation (CF) No.

| Ingredient | 1 | 2 |
|---|---|---|
| Natural extract | — | — |
| Antiperspirant | AP1 | AP2 |
| Cyclomethicone | ✓ | ✓ |
| Aluminum starch | ✓ | ✓ |
| octenylsuccinate | | |
| Phenyltrimethicone | ✓ | ✓ |
| PPG-14 butyl ether | ✓ | ✓ |
| Fumed silica | ✓ | ✓ |
| Hydrogenated castor oil | ✓ | ✓ |
| Stearyl alcohol | ✓ | ✓ |

In Table 2 above, AP1 is aluminum chlorohydrate and AP2 is aluminum zirconium tetrachlorohydrate glycine (AZG). Formulation CF1 included 25.00 wt % AP1 antiperspirant and CF2 included 26.67 wt % AP2 antiperspirant.

The amount for each of cyclomethicone, aluminum starch octenylsuccinate, phenyltrimethicone, PPG-14 butyl ether, fumed silica, hydrogenated castor oil, and stearyl alcohol, in formulations (CF1 and CF2) is as follows.

Formulation CF1 included 49.35 wt. % cyclomethicone, 6.91 wt. % aluminum starch octenylsuccinate; 5.92 wt. % phenyl trimethicone, 4.93 wt. % PPG-14 butyl ether, 2.96 wt. % fumed silica, 2.96 wt. % hydrogenated castor oil, and 1.97 wt. % stearyl alcohol. Formulation CF2 included 48.26 wt. % cyclomethicone, 6.75 wt. % aluminum starch octenylsuccinate; 5.79 wt. % phenyl trimethicone, 4.82 wt. % PPG-14 butyl ether, 2.89 wt. % fumed silica, 2.89 wt. % hydrogenated castor oil, and 1.93 wt. % stearyl alcohol.

### Roll-On Antiperspirant Product

The method of preparing a roll-on product is set out below.
1. Mix cyclomethicone, phenyl trimethicone, stearyl alcohol, PPG-14 butyl ether, and hydrogenated castor oil.
2. Heat to 82°° C. to melt the solids.
3. While maintaining a temperature of 82° C., slowly add fumed silica using a high-speed disk type dispenser.
4. Once all silica is added, increase mixing speed so that peripheral velocity is almost 4000 ft/minute or higher for satisfactory dispersion.
5. Add aluminum starch octenylsuccinate followed by the Natural AP powder. Each component is dispersed uniformly before adding the next.
7. Once all components are added, cool the mixture to 55° C.

### In Vivo Test Procedure

### Study Design

The formulation examples were tested in a standard hot room assessment clinical study. The study panellists underwent thermal stress after application of the antiperspirant compositions. The sweat reduction was determined by gravimetric collection of the sweat.

Subjects: 40 female panellists.

Conditions: 17-day washout period in which the panellists did not use any antiperspirants.

Test Design: Subjects underwent 5-day application period where each was given 400 mg±50 mg of one of the formulation examples. After the 5th day, sweat collection was conducted after the panellists entered the hot room (100° F.±2° F. and 35%±5% relative humidity) for 80 minutes to induce sweating.

Analysis of Data: The data were transformed to their natural logarithms and the averages were analyzed according to the method described by Murphy and Levine (Reference 2). The analysis of covariance model included factors for sequence, subjects within a sequence, test articles, axilla and baseline as the covariate. The date is provided below.

| Formulation | Timepoint | Mean | Mean Diff | % Change | p-value |
|---|---|---|---|---|---|
| 1 | Baseline | 0.61 | | | |
| | 8 Hours | 0.40 | −0.21 | −33.88% | 0.02 |
| 2 | Baseline | 0.64 | | | |
| | 8 Hours | 0.39 | −0.25 | −39.59% | 0.02 |
| 3 | Baseline | 0.79 | | | |
| | 8 Hours | 0.36 | −0.44 | −55.04% | 0.01 |

For each formulation, the % Change in sweat reduction was determined. The clinical study data showed that all of the formulations were effective at reducing sweat, with formulations F1 to F3 showing a sweat reduction greater than 30% when compared to baseline. A sweat reduction of at least 20% (as measured according to US FDA Guidelines) is considered to be suitable for commercial use as an antiperspirant agent.

Formulation F3 showed sweat efficacy results that are parity results when compared to the comparative products with aluminum or aluminum zirconium.

### Examples 4 and 5

Further experiments were performed using Formulation 3 (see Table 1 above), except that the amount of trans-cinnamic acid was varied. Formulation 4 contained 3 wt. % of trans-cinnamic acid; Formulation 5 contained 10 wt. % of trans-cinnamic acid. These formulations were prepared using the same method for manufacturing roll-on antiperspirant products given above.

### In Vivo Tests

In vivo tests were performed using each formulation.

For inclusion in the studies, participants needed to show an amount of sweat of 150 mg in each armpit in an initial assessment. The same methodology as set out above was used to perform these tests, except for the following details.

Formulation 4: 30 panellists participated in the study and during the 5-day application period, each panellist was given 400 mg of the formulation.

Formulation 5: 33 panellists participated in the study and were subjected to an 18-day washout period. Each panellist was given 400 mg of the formulation.

When in the study there were pre-treatment records (baseline) in the evaluation of armpit sweating after the last application, the ratio of the treated armpit with the investigational product adjusted to the ratio of the right-to-left armpit sweating rate is defined for each individual by the formula:

$$Z = ( PC \times T ) / ( PT \times C )$$

where Z is the adjusted ratio from left to right; PC is the amount of sweat obtained in the pre-treatment for the control armpit; PT is the amount of sweat obtained in the pre-treatment for the armpit that will receive the treatment; T is the amount of sweat obtained for the armpit with the formulation of the invention; and C is the amount of sweat obtained for the control armpit.

The results are shown in Tables 3 and 4 below.

TABLE 3
Formulation No. 4
Sweating Values, 8 hours

| | Armpit pre- | Armpit pre- | | | | |
|---|---|---|---|---|---|---|
| | treatment to be | treatment | Treated | Control | | |
| | treated (PT) | control (PC) | Armpit (T) | Armpit (C) | | |
| | (mg) | (mg) | (mg) | (mg) | Z | Z − 0.8 |
| Mean | 1832.7 | 1739.3 | 943.3 | 2227.0 | 0.37 | −0.43 |
| Median | 1790.0 | 1640.0 | 775.0 | 2035.0 | 0.32 | −0.48 |
| Minimum | 1310.0 | 1010.0 | 400.0 | 1630.0 | 0.16 | −0.64 |
| Maximum | 2720.0 | 2950.0 | 4920.0 | 4730.0 | 1.00 | 0.20 |
| Standard error | 68.7 | 86.5 | 150.3 | 105.8 | 0.03 | 0.03 |
| IC 95% | 140.6 | 176.9 | 307.4 | 216.5 | 0.06 | 0.06 |

| | |
|---|---|
| % of research participants with reduction greater than 20% | 96.67 |
| *P-Value | 0.000 |

TABLE 4
Formulation No. 5
Sweating Values, 8 hours

| | Armpit pre- | Armpit pre- | | | | |
|---|---|---|---|---|---|---|
| | treatment to be | treatment | Treated | Control | | |
| | treated (PT) | control (PC) | Armpit (T) | Armpit (C) | | |
| | (mg) | (mg) | (mg) | (mg) | Z | Z − 0.8 |
| Mean | 1849.7 | 1817.5 | 665.8 | 1994.2 | 0.33 | −0.47 |
| Median | 1810.0 | 1850.0 | 650.0 | 2050.0 | 0.34 | −0.46 |
| Minimum | 1200.0 | 1150.0 | 410.0 | 1260.0 | 0.17 | −0.63 |
| Maximum | 3000.0 | 2790.0 | 1090.0 | 2750.0 | 0.49 | −0.31 |
| Standard error | 58.1 | 59.7 | 28.7 | 57.7 | 0.01 | 0.01 |
| IC 95% | 118.4 | 121.7 | 58.5 | 117.6 | 0.03 | 0.03 |

| | |
|---|---|
| % of research participants with reduction greater than 20% | 100.0 |
| *P-Value | 0.000 |

In both studies, no statistically significant differences were found for the median values of sweating between the product and the control. According to the methodology used to evaluate the antiperspirant effect, Formulations 4 and 5 showed a reduction in sweating 8 hours after the last application. Both tests showed that at least 50% of the research participants had a reduction in sweating of at least 20%.

### Example 6

A study to assess the deodorant effect was performed using Formulation 5. The deodorancy study was designed to be a single center, single blind, randomized, trial to determine the deodorancy effectiveness of trans-cinnamic acid.

In this study, 12 panellists were pre-conditioned by using only soap and abstaining from the use of deodorants, antiperspirants, perfumed or medicated products for a minimum of 48 hrs, as in the studies described above.

On the test day, the panellists were evaluated for odor by 3 judges. Odor scores were measured from 0 (no odor) to 4 (severe). To participate in the study, the panellists were required to have an odor score of at least 3.

Immediately after the evaluation and qualification of the panellists, Formulation 5 was applied to one axilla. Randomized application was conducted for all panellists. The panellists were then evaluated for odor at 8 hr, 24 hr and 48 hr time points.

Notably, the results from the study showed that there was a statistically significant decrease in odor scores at 8 hr, 24 hr and 48 hr time points.

### Examples 7 and 8

Formulations 6 and 7 were prepared. Formulation 6 included Emblica officinalis (saberry) extract and trans-cinnamic acid; Formulation 7 included Emblica officinalis (saberry) extract and cinnamon extract.

The natural extracts were obtained from Sabinsa Corporation. The Formulation 6 included 1.5 wt % Emblica officinalis (saberry) and 1.5 wt % trans-cinnamic acid, and the Formulation 7 included 1.5 wt. % Emblica officinalis (saberry) extract and 1.5 wt % cinnamon extract.

In addition, each of Formulations 6 and 7 included the same amount for each of cyclomethicone, aluminum starch succinate, phenyltrimethicone, PPG-14 butyl ether, fumed silica, hydrogenated castor oil, and stearyl alcohol, as follows: 63.82 wt. % cyclomethicone, 7.66 wt. % phenyl trimethicone, 2.55 wt. % stearyl alcohol, 6.38 wt. % PPG-14 butyl ether, 3.83 wt. % hydrogenated castor oil, 3.83 wt. % fumed silica, and 8.93 wt. % aluminum starch octenylsuccinate.

The method of preparing the formulations (6 and 7) as a roll-on antiperspirant product is set out below.
1. Mix cyclomethicone, phenyl trimethicone, stearyl alcohol, PPG-14 butyl ether, and hydrogenated castor oil.
2. Heat to 82° C. to melt the solids.
3. While maintaining a temperature of 82° C., slowly add fumed silica using a high-speed disk type dispenser.
4. Once all silica is added, increase mixing speed so that peripheral velocity is almost 4000 ft/minute or higher for satisfactory dispersion.
5. Add aluminum starch octenylsuccinate followed by the Natural AP powder. Each component is dispersed uniformly before adding the next.
7. Once all components are added, cool the mixture to 55° C.

### Examples 9 and 10

Formulations 8 and 9 were prepared. Formulations 8 and 9 both included trans-cinnamic acid.

The natural extract was obtained from Sabinsa Corporation. Both of Formulations 8 and 9 included 8 wt. % trans-cinnamic acid.

In addition, Formulation 8 included 8 wt. % cyclomethicone, 3 wt. % PPG-14 butyl ether, 2.25 wt. % Steareth 2, 1.25 wt. % Steareth 21, 73 wt. % deionized water, 4 wt. % aluminum zirconium complex solution, and 0.5 wt. % Euxyl PE 9010 based on total weight of the formulation. In addition, Formulation 9 included 8 wt. % cyclomethicone, 3 wt. % PPG-14 butyl ether, 3 wt. % Steareth 2, 1 wt. % Steareth 21, 70.5 wt. % deionized water, 6 wt. % aluminum zirconium complex solution, and 0.5 wt. % Euxyl PE 9010 based on total weight of the formulation.

The method of preparing the formulations as a roll-on antiperspirant product is set out below.
1. Mix cyclomethicone, PPG-14 butyl ether, Steareth 2, and Steareth 21.
2. Heat to 60° C. to melt the solids.
3. Maintain temperature at 60° C.
4. Heat deionized water to 65° C. and add to the heated mixture in step 2 (i.e., the four ingredients) with agitation to emulsify.
5. Cool to the mixture to 50° C. while stirring continuously.
6. Add trans-cinnamic acid and aluminum zirconium salt solution to the mixture and mix well until uniform.
7. Add EuxyIPE9010 and continue mixing.
8. Mix until uniform.

### Examples 11, 12 and 13

Formulations 10, 11 and 12 were prepared. Formulations 10, 11 and 12 included trans-cinnamic acid.

The natural extract was obtained from Sabinsa Corporation. The Formulations 10, 11 and 12 included 10 wt. % trans-cinnamic acid.

In addition, each of Formulations 10 and 11 included the same amount of the following additives: 34.13 wt. % cyclomethicone, 17 wt. % C₁₂-C₁₅ alkyl benzoate, 2.1 wt. % PVM/MA decadiene crosspolymer, 19.2 stearyl alcohol, 4.27 wt. % hydrogenated castor oil, 1 wt. % PEG-8 distearate, 2.3 wt. % bis-stearyl dimethicone, 10 wt. % aluminum chlorohydrate (ACH) powder and qs fragrance. Formulation 12 included 38.4 wt. % cyclomethicone, 19.2 wt. % C₁₂-C₁₅ alkyl benzoate, 2.4 wt. % PVM/MA decadiene crosspolymer, 21.6 stearyl alcohol, 4.8 wt. % hydrogenated castor oil, 1.2 wt. % PEG-8 distearate, 2.4 wt. % bis-stearyl dimethicone, 10 wt. % aluminum chlorohydrate (ACH) powder and 0 fragrance.

The method of preparing the formulation as a stick antiperspirant product is set out below.
1. Mix all ingredients except the trans-cinnamic acid and fragrance.
2. Heat to 82° C. to melt the solids.
3. Add trans-cinnamic acid with continuous mixing.
3. While maintaining a temperature of 82° C., mix well to disperse solids and then begin cooling.
4. Add fragrance at 60° C. and mix well.
5. Pour into stick containers at 52-55° C. and cool completely.

### Example 14

Formulation 13 was prepared. Formulation 13 included trans-cinnamic acid.

The natural extract was obtained from Sabinsa Corporation. Formulation 13 included 5.5 wt. % trans-cinnamic acid.

In addition, Formulation 13 included 5.2 wt. % cyclomethicone, 2.3 wt. % isopropyl myristate, 1.8 wt. % Bentone Gel VS 5PC V (Bentone), 0.2 wt. % fragrance, and 85 wt. % hydrocarbon propellant based on total weight of the formulation.

The method of preparing the formulations as an aerosol antiperspirant product is set out below.
1. Combine together cyclomethicone, isopropyl myristate, and Bentone in a vessel until smooth and homogenous with an overhead mixer equipped with turbine blades (10-15 min/300 rpm).
2. Slowly add trans-cinnamic acid and continue mixing until homogenous (10-15 min).
3. Add propellant, and mix for 5 minutes.
4. Homogenize concentrate at 5000 rpm for 10 minutes.
5. Fill concentrate in aerosol cans and charge with propellant.

### Example 15

A conditioner formulation containing trans-cinnamic acid according to certain embodiments of the invention was prepared as follows.

| Phase | Materials: | Weight (%) | Weight (g) |
|---|---|---|---|
| A | Deionized water | 81.9 | 81.9 |
| A | Butylene Glycol | 2.5 | 2.5 |
| A | Caprylyl Glycol (and) Caprylhydroxamic | | |
| | Acid (and) glycerine | 0.9 | 0.9 |
| A | Trans Cinnamic Acid | 1 | 1 |
| A | Calcium Pantothenate | 0.2 | 0.2 |
| B | Cetearyl Alcohol (and) Behentrimonium | | |
| | Methosulfate | 5 | 5 |
| B | Crambe Abyssinica Seed Oil | 3 | 3 |
| B | Isopropyl Palmitate | 1 | 1 |
| C | Cetearyl Alcohol | 1.5 | 1.5 |
| C | Cyclopentasiloxane | 2 | 2 |
| C | PEG-2 Dimeadowfoamamidoethylmonium | 1 | 1 |
| | Methosulfate | | |
| | TOTAL | 100.00 | 100.00 |

Phase A materials were combined and heated to 80° C. with propeller mixing.
Phase B materials were combined and heated to 80° C. and added to the heated Phase A materials with propeller mixing.
The mixture of Phase A and B materials was cooled to 45° C. with mixing.
Phase C materials were added to the Phase A and B mixture.
The Phase A, B, and C mixture was cooled to 30° C.
The cooled Phase A, B and C mixture was then packaged.

### Example 16

A conditioner formulation containing trans cinnamic acid according to certain embodiments of the invention was prepared as follows.

| Phase | Materials: | Weight (%) | Weight (g) |
|---|---|---|---|
| A | Deionized water | 56.55 | 56.55 |
| A | Disodium EDTA | 0.1 | 0.1 |
| A | Panthenol | 0.1 | 0.1 |
| A | Trans Cinnamic Acid | 1 | 1 |
| B | Sodium Laureth Sulfate | 23 | 23 |
| B | Coocamidopropyl Betaine | 7.5 | 7.5 |
| B | Coco-Glucoside | 5 | 5 |
| B | Cocomide MIPA | 1.5 | 1.5 |
| B | PEG-150 Pentaerythrityl Tetrastearate | 1 | 1 |
| B | Glycol Distearate (and) Laureth-4 (and) | | |
| | Cocamidopropyl Betaine | 2 | 2 |
| C | PEG-2 Dimeadowfoamamidoethylmonium | | |
| | Methosulfate | 2 | 2 |
| C | Methylchloroisothiazolinone (and) | | |
| | Methylisothiazolinone | 0.05 | 0.05 |
| C | Fragrance | 0.2 | 0.2 |
| | TOTAL | 100.00 | 100.00 |

Phase A materials were combined and heated to 80° C. with propeller mixing.
Phase B materials were combined and heated to 80° C. and added to the heated Phase A materials with propeller mixing.
The mixture of Phase A and B materials was cooled to 40° C. with mixing.
Phase C materials were added to the Phase A and B mixture.
The Phase A, B, and C mixture was cooled to 30° C.
The cooled Phase A, B and C mixture was then packaged.

### REFERENCES

The following documents are referenced in the description.
1. A. P. Rauter et al.; “Nomenclature of flavonoids (IUPAC Recommendations 2017)”; Pure Appl. Chem., 90 (9), (2018), 1429-1486.
2. T. D. Murphy & M. J. Levine; “Analysis of Antiperspirant Efficacy Test Results”; Journal of the Society of Cosmetic Chemists, 42, (1991), 167-197.
3. “Guidelines for Effectiveness Testing of OTC Antiperspirant Drug Products”, published by the US FDA, when read in conjunction with § 350.60 (21 CFR 350.60) of the final monograph (final rule) for OTC antiperspirant drug products, published in the US FEDERAL REGISTER on June 9, 2003 (68 FR 34273).

The entire disclosure of each document cited herein is hereby incorporated by reference. All references cited in this disclosure are incorporated by reference to the same extent as if each reference had been incorporated by reference in its entirety individually.

The citation and incorporation of patent documents herein is done for convenience only and does not reflect any view of the validity, patentability, and/or enforceability of such patent documents.

A number of embodiments of the disclosure have been described. Nevertheless, it will be understood that various modifications may be made without departing from the spirit and scope of the present disclosure. Accordingly, other embodiments are within the scope of the following claims.

All headings and sub-headings are used herein for convenience only and should not be construed as limiting the invention in any way.

We claim:

1. A personal care composition, comprising:
a personal care agent, comprising:
an optionally substituted benzenepropenoic acid represented by formula (I): 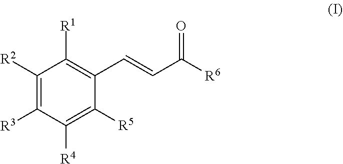
wherein each of R¹ to R⁵ is independently selected from hydrogen, hydroxy and C₁-C₆alkoxy, and R⁶ is selected from hydrogen and hydroxy; and
a carrier.

2. The personal care composition according to claim 1, wherein the optionally substituted benzenepropenoic acid, comprises:
a phytochemical, or
a material derived or obtained from the phytochemical, or
an aluminum-free synthetic/chemical material that is an equivalent of the phytochemical or material derived or obtained from the phytochemical.

3. The personal care composition according to claim 1, wherein the optionally substituted benzenepropenoic acid is selected from cinnamic acid and its isomers, o-coumaric acid, m-coumaric acid, p-coumaric acid, caffeic acid, ferulic acid, 5-hydroxyferulic acid and sinapic acid.

4. The personal care composition according to claim 3, wherein the optionally substituted benzenepropenoic acid is selected from cinnamon extract and trans-cinnamic acid.

5. The personal care composition according to claim 4, wherein the optionally substituted benzenepropenoic acid comprises from about 1.5 to about 10.0 wt. %, based on total weight of the personal care composition.

6. The personal care composition of claim 4, further comprising a combination or blend of cinnamon extract or trans-cinnamic acid, and Emblica officinalis extract.

7. The personal care composition according to claim 6, wherein the cinnamon extract comprises cinnamic acid.

8. The personal care composition according to claim 1, wherein the carrier is a topically acceptable carrier.

9. The personal care composition according to claim 1, wherein the personal care agent comprises an agent selected from an antiperspirant agent, a deodorant agent, an astringent agent, and an anti-microbial agent.

10. The personal care composition according to claim 1, wherein said composition is in a form selected from a cream, a gel, a paste, a liquid, a lotion, a foam, an emulsion, a scrub and a powder.

11. The personal care composition according to claim 1, further comprising one or more additives selected from an emollient, a fragrance, a thickener, and a preservative.

12. A method of preparing a personal care composition, comprising:
preparing a mixture, comprising:
a personal care agent, comprising:
an optionally substituted benzenepropenoic acid represented by formula (I): 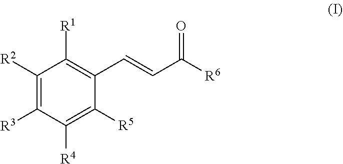
wherein each of R¹ to R⁵ is independently selected from hydrogen, hydroxy and C₁-C₆ alkoxy, and R⁶ is selected from hydrogen and hydroxy; and
a carrier.

13. The method of claim 12, wherein the optionally substituted benzenepropenoic acid, comprises:
a phytochemical, or
a material derived or obtained from the phytochemical, or
an aluminum-free synthetic/chemical material that is an equivalent of the phytochemical or material derived or obtained from the phytochemical.

14. The method of claim 12, wherein the mixture further comprises one or more materials selected from lubricant, fatty alcohol, thickener and starch.

15. The method of claim 14, wherein the step of preparing the mixture, comprises:
combining the lubricant, fatty alcohol and acceptable carrier to form a first mixture;
heating the first mixture to form a heated first mixture;
adding thickener to the heated first mixture to form a second mixture; and
adding starch to the second mixture, followed by adding the personal care agent.

16. A personal care composition, comprising:
a personal care agent, comprising:
trans-cinnamic acid, Emblica officinalis extract and mixtures or blends thereof, being absent of aluminum or aluminum-zirconium salts; and
a carrier.

17. The personal care composition of claim 16, wherein the personal care agent comprises from 1.5 wt. % to 10.0 wt. % based on the total weight of the personal care composition.

18. The personal care composition of claim 16, wherein said composition further comprises cyclomethicone, starch rheology modifier, phenyl trimethicone, butyl ether, fumed silica, hydrogenated castor oil and stearyl alcohol.

19. The personal care composition of claim 16, wherein the personal care agent comprises 3.0 wt. % of trans-cinnamic acid.

20. The personal care composition of claim 16, wherein the personal care agent comprises a mixture or blend of 1.5 wt. % of trans-cinnamic acid and 1.5 wt. % of Emblica officinalis extract.
